(19) United States
(12) Patent Application Publication
(10) Pub. No.: US 2019/0069881 A1
(43) Pub. Date: Mar. 7, 2019

(54) ULTRASOUND IMAGE DIAGNOSIS APPARATUS, MEDICAL IMAGE PROCESSING APPARATUS, AND COMPUTER PROGRAM PRODUCT

(71) Applicant: Canon Medical Systems Corporation, Otawara-shi (JP)

(72) Inventors: Yasunori OHSHIMA, Yaita (JP); Tomoko SUZUKI, Nasushiobara (JP); Taku MURAMATSU, Otawara (JP); Katsuyuki TAKAMATSU, Yaita (JP)

(73) Assignee: Canon Medical Systems Corporation, Otawara-shi (JP)

(21) Appl. No.: 16/121,094

(22) Filed: Sep. 4, 2018

(30) Foreign Application Priority Data
Sep. 4, 2017 (JP) 2017-169864

(51) Int. Cl. A61B 8/00; A61B 8/14; A61B 8/08

(52) U.S. Cl. CPC A61B 8/463; A61B 8/14; A61B 8/5223; A61B 8/08; A61B 8/467

(57) ABSTRACT

According to one embodiment, an ultrasound image diagnosis apparatus includes a display, an input circuit, and processing circuitry. The display is configured to display an ultrasound image generated in response to a reflected signal of ultrasound waves transmitted toward a target site to be diagnosed from an ultrasound probe. The input circuit includes a first button that is used to set a start point as a measurement start position and an end point as a measurement end position upon measuring the size of a measurement object on the ultrasound image displayed on the display. The processing circuitry is configured to cause the display to display the ultrasound image, and determine whether the end point is set with the first button or with a second button other than the first button.

### CROSS-REFERENCE TO RELATED APPLICATION

This application is based upon and claims the benefit of priority from Japanese Patent Application No. 2017-169864, filed on 2017 Sep. 4; the entire contents of which are incorporated herein by reference.

### FIELD

Embodiments described herein relate generally to an ultrasound image diagnosis apparatus, a medical image processing apparatus, and a computer program product.

### BACKGROUND

In recent years, a subject is sometimes examined with a modality that collects information on the inside of the subject and generates a medical image by imaging the inside of the subject based on the information. Examples of the modality include ultrasound diagnosis apparatuses. The ultrasound image diagnosis apparatus receives reflected signals of ultrasound waves transmitted toward a target site to be diagnosed, and generates an ultrasound image relating to the target site.

Then, the user measures the size of the target site depicted in the ultrasound image while observing the image. This measurement is a process necessary to obtain useful information in, for example, diagnosing fetal development or examining the heart.

In such measurement process, there are cases where the academic society recommends measuring the same measurement object a plurality of times depending on the object. As described above, the measurement result is information required for diagnosis, and the diagnosis may vary depending on the measurement result. Therefore, it is better to measure the measurement object not only once but twice or more to reduce the occurrence of errors as much as possible.

To perform a measurement, for example, to measure a distance, first, a start point is set as a position to start the measurement. After that, an end point (a position to end the measurement) is set. By setting the start point and the end point, the distance between them is measured. Because of the significance of the measurement process as described above, the user considers the positions of the start point and the end point for measurement to set them.

However, the start point and the end point, especially the end point, may sometimes be set to an unintended position depending on the operation of the user. When the measurement is performed with the unintended position as the end point, the measurement result is likely to be inappropriate.

The user may be confused by the inappropriate result and hesitant to use it as information to make diagnosis. Further, such measurement result may lead to inappropriate diagnosis results.

### BRIEF DESCRIPTION OF THE DRAWINGS

FIG. 1 is a perspective view illustrating the appearance of an ultrasound image diagnosis apparatus according to an embodiment;

FIG. 2 is a functional block diagram illustrating the overall configuration of the ultrasound image diagnosis apparatus of the embodiment;

FIG. 3 is a diagram illustrating an example of an input circuit of the ultrasound image diagnosis apparatus of the embodiment;

FIG. 4 is a table for explaining the operation of a control circuit using a measurement result calculation function according to the embodiment;

FIG. 5 is a table for explaining the operation of the control circuit using the measurement result calculation function according to the embodiment;

FIG. 6 is a flowchart illustrating user's operation to measure a measurement object according to the embodiment; and

FIG. 7 is a flowchart illustrating user's operation to measure the measurement object according to the embodiment.

### DETAILED DESCRIPTION

In general, according to one embodiment, an ultrasound image diagnosis apparatus includes a display, an input circuit, and processing circuitry. The display is configured to display an ultrasound image generated in response to a reflected signal of ultrasound waves transmitted toward a target site to be diagnosed from an ultrasound probe. The input circuit includes a first button that is used to set a start point as a measurement start position and an end point as a measurement end position upon measuring the size of a measurement object on the ultrasound image displayed on the display. The processing circuitry is configured to cause the display to display the ultrasound image, and determine whether the end point is set with the first button or with a second button other than the first button.

Exemplary embodiments will be described in detail with reference to the drawings. In the following, an ultrasound image diagnosis apparatus will be described as an example; however, the embodiments are applicable to any other modalities than the ultrasound image diagnosis apparatus or medical image processing apparatuses such as work stations as long as they can measure the size of a measurement object by using a medical image generated based on information relating to a target site of a subject to be diagnosed.

### [Configuration of Ultrasound Image Diagnosis Apparatus]

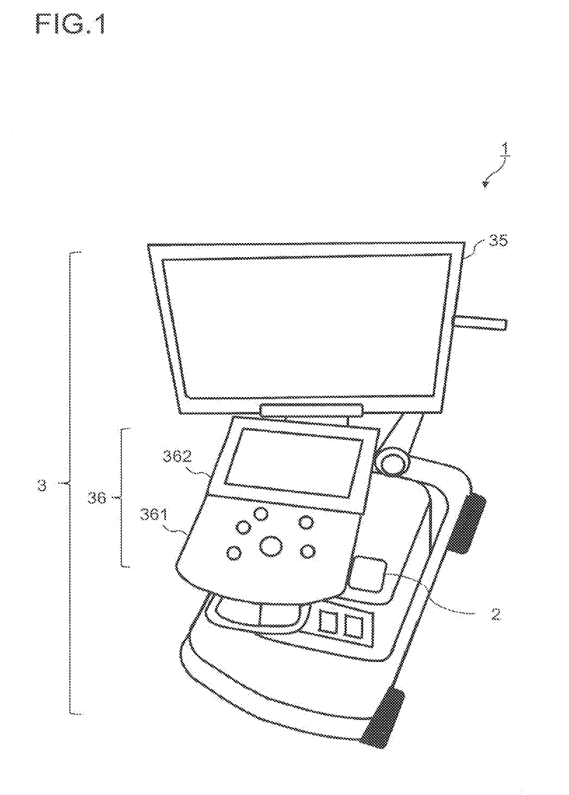

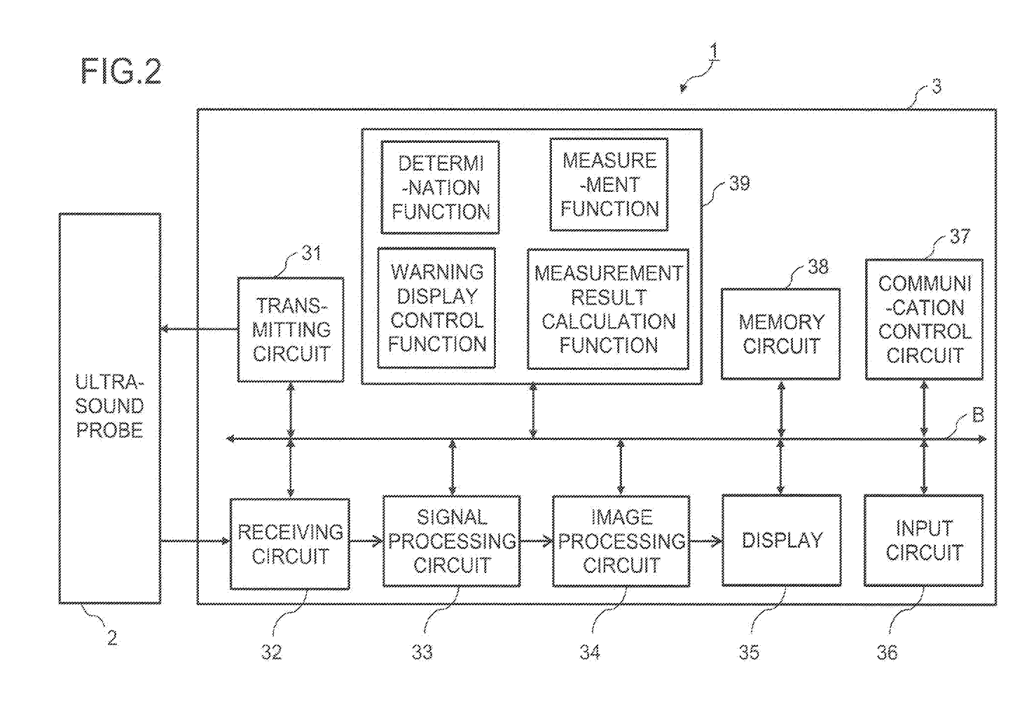

FIG. 1 is a perspective view illustrating the appearance of an ultrasound image diagnosis apparatus 1 according to an embodiment. FIG. 2 is a functional block diagram illustrating the overall configuration of the ultrasound image diagnosis apparatus 1 of the embodiment. As illustrated in FIG. 1, the ultrasound image diagnosis apparatus 1 includes an ultrasound probe 2 configured to transmit and receive ultrasound waves to and from a subject, and a main body 3. The ultrasound probe 2 is detachably connected to the main body 3.

The ultrasound image diagnosis apparatus 1 is an example of a medical image diagnosis apparatus that is capable of noninvasively examining a structure inside the subject, the blood flow state, and the like. The ultrasound image diagnosis apparatus 1 is configured to transmit ultrasound waves toward the inside of a subject from the ultrasound probe 2 having transducers (piezoelectric transducers) at the tip, and receive reflected waves caused by acoustic impedance mismatch inside the subject through the transducers of the ultrasound probe 2. The ultrasound image diagnosis apparatus 1 generates an ultrasound image based on the received signal.

The ultrasound probe 2 is configured to transmit ultrasound waves into the subject through each of the ultrasound transducers to scan a scan area, and receive reflected waves from the subject as echo signals. Examples of the scan include various types of scans such as B mode scan and Doppler mode scan. Besides, examples of the ultrasound probe 2 include a sector scan probe, a linear scan probe, a convex scan probe, and the like, and one of them is arbitrarily selected according to the site to be diagnosed.

Although the ultrasound image diagnosis apparatus 1 is described as including the ultrasound probe 2 in this embodiment, the ultrasound probe 2 is not an essential constituent element. Therefore, the ultrasound image diagnosis apparatus 1 need not necessarily include the ultrasound probe.

The main body 3 includes a transmitting circuit 31, a receiving circuit 32, a signal processing circuit 33, an image processing circuit 34, a display 35, and an input circuit 36. The transmitting circuit 31 is configured to transmit a drive signal to the ultrasound probe 2. The receiving circuit 32 is configured to receive echo signals from the ultrasound probe 2. The signal processing circuit 33 is configured to process the echo signals. The image processing circuit 34 is configured to generate an ultrasound image. The display 35 is configured to display the ultrasound image and also, for example, calipers or the like used for measuring the size of a measurement object superimposed on the ultrasound image. The input circuit 36 is configured to receive an input signal as being operated by the user such as an examiner.

The main body 3 further includes a communication control circuit 37 configured to control the exchange of signals with other devices (not illustrated), a memory circuit 38, and a control circuit 39 configured to control each part. These circuits are connected to a bus B and can exchange various signals. The functions of each of the circuits are described below in further detail.

Under the control of the control circuit 39, the transmitting circuit 31 generates a drive signal for causing the ultrasound probe 2 to generate ultrasound waves, i.e., an electric pulse signal (hereinafter referred to as “drive pulse”) to be applied to each of the piezoelectric transducers. The transmitting circuit 31 transmits the drive pulse to the ultrasound probe 2. The transmitting circuit 31 includes circuits such as, for example, a reference pulse generating circuit, a delay control circuit, a drive pulse generating circuit, and the like (not illustrated), and those circuits perform the functions mentioned above.

The receiving circuit 32 receives an echo signal, i.e., received signal from the ultrasound probe 2. The receiving circuit 32 performs phasing addition on the received signal, and outputs the resultant signal to the signal processing circuit 33.

The signal processing circuit 33 generates various types of data using the received signal from the ultrasound probe 2 fed by the receiving circuit 32, and outputs the data to the image processing circuit 34 and the control circuit 39. The signal processing circuit 33 includes, for example, a B mode processing circuit (or Bc mode processing circuit), a Doppler mode processing circuit, a color Doppler mode processing circuit, and the like (not illustrated). The B mode processing circuit visualizes amplitude information of the received signal, and generates data based on a B mode signal. The Doppler mode processing circuit extracts Doppler shift frequency component from the received signal, and applies fast Fourier transform (FFT) or the like thereto, thereby generating Doppler signal data of blood flow information. The color Doppler mode processing circuit visualizes the blood flow information based on the received signal, and generates data based on a color Doppler mode signal.

The image processing circuit 34 generates two-dimensional or three-dimensional ultrasound images related to the scan area based on the data supplied from the signal processing circuit 33. For example, the image processing circuit 34 generates volume data related to the scan area from the data supplied. Then, from the volume data generated, the image processing circuit 34 generates data of a two-dimensional ultrasound image by multi-planar reconstruction (MPR) or data of a three-dimensional ultrasound image by volume rendering. The image processing circuit 34 outputs the two-dimensional or three-dimensional ultrasound image to the display 35. Examples of the ultrasound image include a B mode image, a Doppler mode image, a color Doppler mode image, an M mode image, and the like.

The display 35 displays various images such as the ultrasound image generated by the image processing circuit 34 and an operation screen (e.g., graphical user interface (GUI) configured to receive various instructions from the user) under the control of the control circuit 39. The display 35 is also capable of displaying calipers to be arranged at the start point and the end point for measurement, measurement results, and the like. As the display 35, for example, a liquid crystal display (LCD), an organic electroluminescence (EL) display, or the like can be used.

The input circuit 36 receives various input operations made by the operator to provide, for example, an instruction to select a measurement item, set the start point and the end point of a measurement object, display an image or switch images, designation of the mode, various settings, and the like. For example, GUI, input devices such as buttons, a keyboard, a trackball, a touch panel displayed on the display 35, or the like can be used as the input circuit 36.

Incidentally, in the embodiment, the display 35 and the input circuit 36 are each described as one constituent element of the ultrasound image diagnosis apparatus 1 as illustrated in FIGS. 1 and 2; however, it is not so limited. The display 35 need not necessarily be a constituent element of the ultrasound image diagnosis apparatus 1, but may be separated from the ultrasound image diagnosis apparatus 1.

Further, the input circuit 36 may be a touch panel displayed on the separate display. In the embodiment, as illustrated in the view of the ultrasound image diagnosis apparatus 1 in FIG. 1, the input circuit 36 includes a panel 361 and a screen 362. The panel 361 is provided with buttons, a trackball, and the like. The screen 362 is a touch panel, and when the user performs an operation such as tapping on a button displayed, a function assigned to the button is activated.

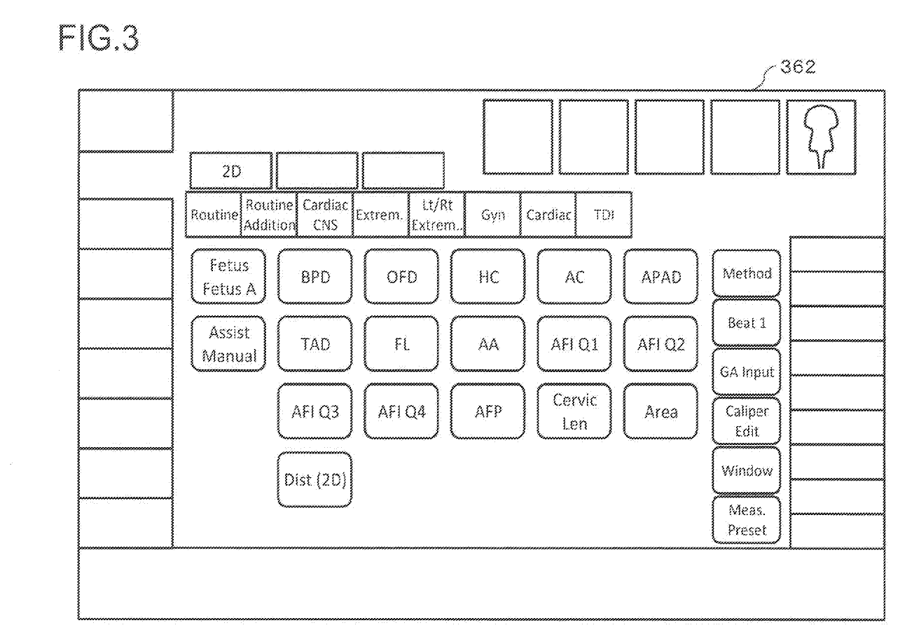

FIG. 3 illustrates an example of the screen 362 as the input circuit of the ultrasound image diagnosis apparatus 1 of the embodiment. On the screen, a plurality of buttons are displayed in a plurality of areas. Each of the buttons is arranged in one of the areas depending on the function assigned thereto. For example, five large buttons lined up on the upper right of the screen each correspond to a type of ultrasound probe used to acquire internal information of the subject for generating an ultrasound image displayed on the display 35. In FIG. 3, the display indicates that only one ultrasound probe is active.

In addition, a plurality of buttons are displayed in the center area of the screen. For example, “Routine” button is displayed on the leftmost side in the upper part of the center area, and seven buttons are displayed on the right side thereof. These buttons are used for selecting whether it is a routine work or another to photograph a target site to be diagnosed with the ultrasound image diagnosis apparatus 1. Actual measurement items displayed below the buttons vary depending on which button is selected.

On the right side of the center area of the screen, there are provided buttons for selecting a method of performing a measurement, for example, setting of calipers. Further, buttons relating to measurement items are arranged so as to be surrounded by the above-described group of buttons in the center area of the screen. In the example of FIG. 3, measurement items such as BPD (biparietal diameter) and OFD (occipital-frontal diameter) are displayed. When the user selects a measurement item displayed on the screen 362, the measurement process related to the selected item is started.

While the screen 362 has been described with reference to an example of the screen illustrated in FIG. 3, for example, the arrangement of buttons such as those for measurement items, types of buttons to be displayed, the layout of the screen, and the like can be set arbitrarily. Besides, the screen 362 may be detachable and portable.

The communication control circuit 37 enables the ultrasound image diagnosis apparatus 1 to communicate with, for example, medical image diagnosis apparatuses (modalities), servers, medical image processing apparatuses, and the like (not illustrated) each connected to a communication network (not illustrated). Information and medical images exchanged between the communication control circuit 37 and other devices via the communication network may be in conformity with any standard such as digital imaging and communication in medicine (DICOM) or the like. The connection to the communication network or the like may be either wired or wireless.

The memory circuit 38 is formed of, for example, a semiconductor or a magnetic disk. The memory circuit 38 stores programs to be executed by the control circuit 39 and data. The memory circuit 38 also stores measurement results, calculation results obtained by using the measurement results, and the like.

In the case of a medical image processing apparatus, measurement of a measurement object, calculation, or warning to the user is performed when the control circuit 39 implements a computer program stored in the memory circuit 38.

The control circuit 39 comprehensively controls each part of the ultrasound image diagnosis apparatus 1. The control circuit 39 causes the display 35 to display the ultrasound image generated by the image processing circuit 34. Besides, the control circuit 39 measures a measurement object by using the ultrasound image displayed on the display 35. Depending on the measurement result, the control circuit 39 notifies the user of a warning.

The control circuit 39 implements the measurement function, determination function, measurement result calculation function, and warning display control function. The measurement function is a function of measuring a measurement object by using the ultrasound image displayed on the display 35. To perform a measurement process, first, the user selects a measurement item. Having selected the measurement item, the user sets the start point as the measurement start position and the end point as the measurement end position through the input circuit 36. When the start point and the end point have been set, the control circuit 39 automatically performs the measurement process.

The control circuit 39 performs the measurement process by the measurement function. The measurement result obtained is stored in, for example, the memory circuit 38. The measurement result may be stored in association with various items that can be set arbitrarily, such as information on measurement items and measurement date and time.

The control circuit 39 detects, for example, that the user touches one of the measurement item buttons displayed on the screen 362, and starts the measurement process with respect to a measurement item assigned to the button touched. The control circuit 39 displays calipers that suits the measurement item selected by the user superimposed on the ultrasound image on the display 35 by using the measurement function.

First, the user set the start point indicating the measurement start position by using the calipers displayed on the display 35. Specifically, the user moves the calipers that indicate the start point to a desired position with the input circuit 36 such as, for example, a mouse or a trackball. Then, the user sets the position of the calipers by, for example, pressing a button on the panel 361. That is, the position set on the ultrasound image is the start point.

The button used to set the calipers to the position of the start point is hereinafter referred to as “first button”. It is assumed herein that the first button is located on the panel 361; however, it may be provided on the screen 362 as well as on the panel 361.

After setting the start point as the measurement start position on the ultrasound image by using the first button, then, the user sets the end point as the measurement end position. In the measurement process, the distance between the start and end points is measured. The user moves the calipers to a desired position as the measurement end position to set the end point.

Generally, the user uses the first button also to set the end point. This is because the first button is preset to be the button used for setting the position of the calipers, i.e., the positions of the start point and the end point.

However, the user may not set the end point with the first button for some reason. For example, there may be a case where the user presses some button to start another process than the measurement process after setting the start point. In other words, the user may press another button such as a print button or a save button after setting the start point. For another example, the user may touch a button indicating a measurement item displayed on the screen 362 instead of pressing the first button.

In such a case, while the user has not pressed the first button, the ultrasound image diagnosis apparatus 1 has received an instruction to shift to another process. Therefore, after setting the end point, the control circuit 39 starts the process according to the received signal to follow the new instruction.

However, in this case, the end point may be set just for convenience in order to shift to the next process. From this viewpoint, the user's intention may not be reflected in the setting of the end point with respect to the position of the end point. In other words, the end point may be set to a position not intended by the user.

If the end point is set to an unintended position, the measurement process is not performed in conformity with the measurement item. Accordingly, the measurement result obtained is highly likely to be inappropriate when considered in relation to the measurement item. If such a measurement result is contained in measurement results obtained by the required number of measurement processes, the user may make an erroneous diagnosis.

For this reason, the control circuit 39 has the determination function to determine whether the setting of the end point has been performed appropriately. The control circuit 39 determines whether the end point has been set to a position intended by the user with the determination function. Specifically, the control circuit 39 determines whether the end point has been set with the first button used for setting the start point.

In other words, the control circuit 39 determines whether the end point has been set by using another button than the first button with the determination function. Hereinafter, all buttons that transmit a signal that triggers the setting of the end point without using the first button are referred to as “second button”. That is, the “second button” is another (different) button than the first button and is a button that the user presses while setting the end point. Therefore, the second button may be a button on either the panel 361 or the screen 362, and all the buttons except the first button can be the second button.

The control circuit 39 has the measurement result calculation function to calculate whether the measurement result indicates an abnormal value. For example, the control circuit 39 calculates the average and variance of a plurality of measurement results.

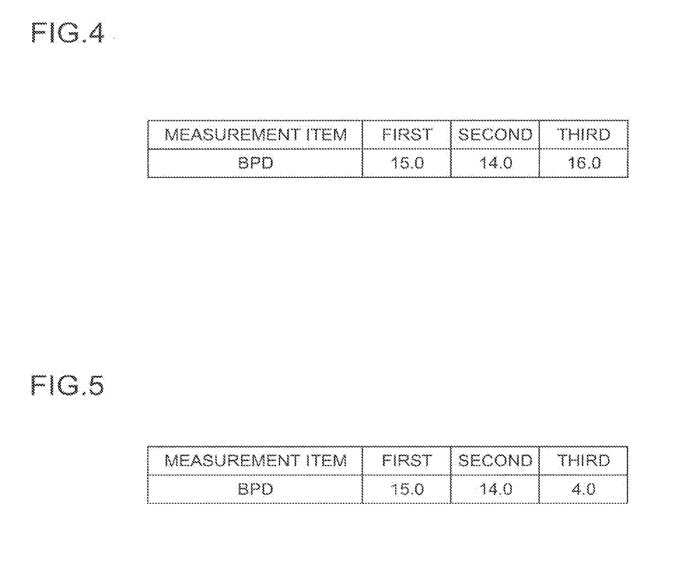

FIGS. 4 and 5 are tables for explaining the operation of the control circuit 39 using the measurement result calculation function according to the embodiment. FIGS. 4 and 5 illustrate the results of measuring “BPD” as a measurement item three times. In the table of FIG. 4, the first measurement result is “15.0”, the second measurement result is “14.0”, and the third measurement result is “16.0”. In the table of FIG. 5, the first measurement result is “15.0”, the second measurement result is “14.0”, and the third measurement result is “4.0”.

The control circuit 39 performs calculation based on the measurement results indicated in FIG. 4 or 5 by using the measurement result calculation function. For example, the control circuit 39 obtains the average value of the measurement results. The average value obtained from the measurement results in FIG. 4 is “15.0”, and the three measurement results are less likely to contain an abnormal value. On the other hand, the average value obtained from the measurement results in FIG. 5 is “11.0”. The average value largely differs from the three measurement results, and it is highly likely that the measurement results contain an abnormal value.

Further, the control circuit 39 may obtain, for example, the variance value by using the measurement result calculation function. For example, the variance value obtained from the measurement results in FIG. 4 is about “0.67”. On the other hand, the variance value obtained from the measurement results in FIG. 5 is about “24.67”. The smaller the variance value, the less spread out the measurement results are. Accordingly, from the calculation results, it is found that the measurement results in FIG. 5 are highly likely to contain an abnormal value. This way of obtaining calculation results is hereinafter referred to as “calculation result acquisition method 1” as appropriate.

The variance value may be obtained with respect to each of the measurement results. For example, the variance value can be obtained with respect to the first and second measurement results, the second and third measurement results, and the third and first measurement results indicated in FIG. 5. This way of obtaining calculation results is hereinafter referred to as “calculation result acquisition method 2” as appropriate.

In the case where the control circuit 39 obtains the variance value of the measurement results in FIG. 5 using only the first and second measurement results without using the third measurement result, the variance value is “0.25”. Meanwhile, when the control circuit 39 obtains the variance value using only the second and third measurement results without using the first measurement result, the variance value is “25.00”. When the control circuit 39 obtains the variance value using only the third and first measurement results without using the second measurement result, the variance value is “30.25”. Looking at the variance values obtained by using the measurement result calculation function of the control circuit 39, the variance value obtained without using the third measurement result is smaller than those obtained using the third measurement result. From this calculation result, it can be determined that the third measurement result is highly likely to be an abnormal value.

The control circuit 39 determines whether the calculation result obtained by the measurement result calculation function indicates an abnormal value by using the determination function. For example, the control circuit 39 compares the calculation result with a threshold value set in advance to determine whether it indicates an abnormal value. For example, when measurements have been performed the number of times determined for each measurement item, the variance value is obtained from the measurement results. Assuming that the threshold value is set to “3”, for example, in the case of the calculation result acquisition method 1, the variance value obtained as the calculation result from the measurement results of “BPD” in FIG. 4 is “0.67”, which is smaller than the threshold value. Thus, the control circuit 39 determines that acceptable measurement results have been obtained from any of the measurements.

On the other hand, as to the measurement results in FIG. 5, the variance value obtained as the calculation result is “24.67”, which is larger than the threshold value. Accordingly, the control circuit 39 determines that one of the measurement results is an abnormal value. That is, the control circuit 39 determines that an abnormal value is contained in the measurement results obtained for the measurement item “BPD”. The threshold value can be arbitrarily set for each measurement item.

In the case of the calculation result acquisition method 2, it is possible to indicate which measurement result is an abnormal value more accurately. For example, using the measurement results in FIG. 5, the control circuit 39 obtains the variance value with respect to each pair of the first and second measurement results, the second and third measurement results, and the third and first measurement results. The variance value of each pair is as described above, and the variance value obtained without using the third measurement result is smaller than those obtained using the third measurement result. From this, it can be determined that the third measurement result is highly likely to be an abnormal value. Thus, the measurement result that indicates an abnormal value can be figured out more finely.

The control circuit 39 has the warning display control function to notify the user of an abnormal value, if any, in the measurement results.

The warning is notified by, for example, displaying the calculation result with respect to a measurement item containing an abnormal value in a different color on the display 35. For example, when the calculation result acquisition method 1 is used, the field of a measurement item containing an abnormal value is displayed in a color different from the color of the field of a measurement item not containing an abnormal value. When the calculation result acquisition method 2 is used, the field of a measurement result that indicates an abnormal value is displayed in a different color.

Regarding the warning display, in addition to the method of notifying the user visually as described above, any other method can be used as long as it can appeal to any of the five senses of the user.

Incidentally, the measurement function, determination function, measurement result calculation function, and warning display control function of the control circuit 39 can be realized by a computer program that is executed by a processor and stored in a predetermined memory, the memory circuit 38, or the like. The term “processor” as used herein refers to a circuit such as, for example, a dedicated or general central processing unit (CPU) arithmetic circuit (circuitry), an application specific integrated circuit (ASIC), a programmable logic device such as a simple programmable logic device (SPLD) and a complex programmable logic device (CPLD), a field programmable gate array (FPGA), or the like.

The processor reads out, for example, a program stored in the memory circuit 38 or directly incorporated in the circuit of the processor and executes it, thereby realizing the functions. Each processor may be provided with a recording circuit for storing the program. The recording circuit may store, for example, a program corresponding to the functions of the signal processing circuit 33 illustrated in FIG. 1, or may have the configuration of the memory circuit 38 illustrated in FIG. 1. The memory circuit is formed of a storage device, examples of which include a semiconductor memory and a magnetic disk such as a general random access memory (RAM) and a hard disc drive (HDD).

### [Operation]

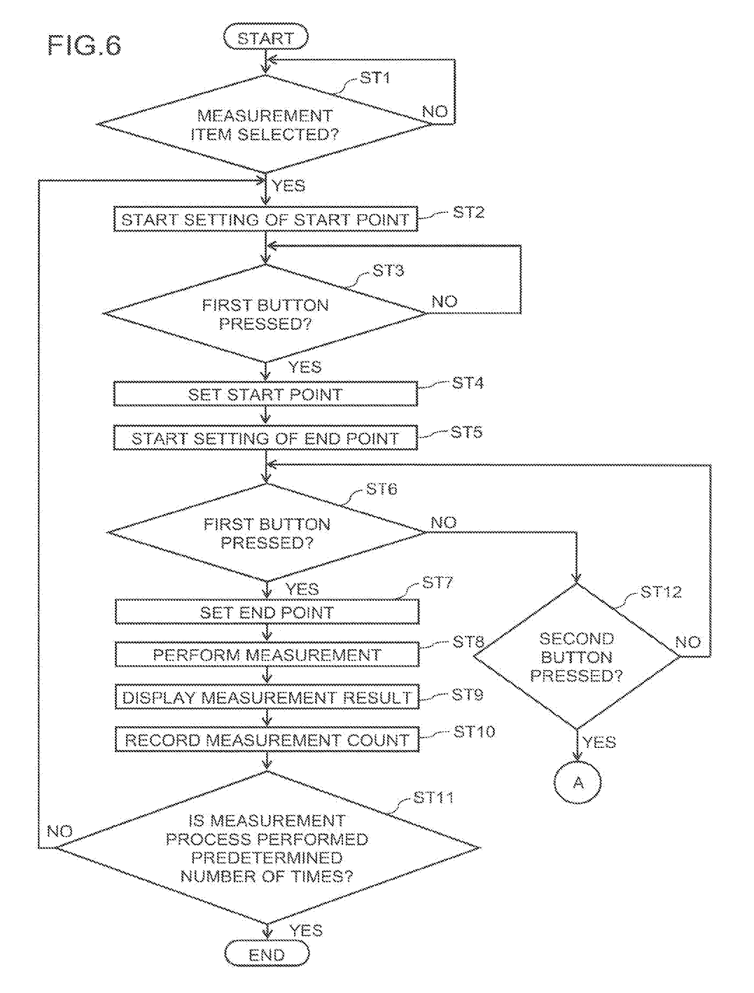

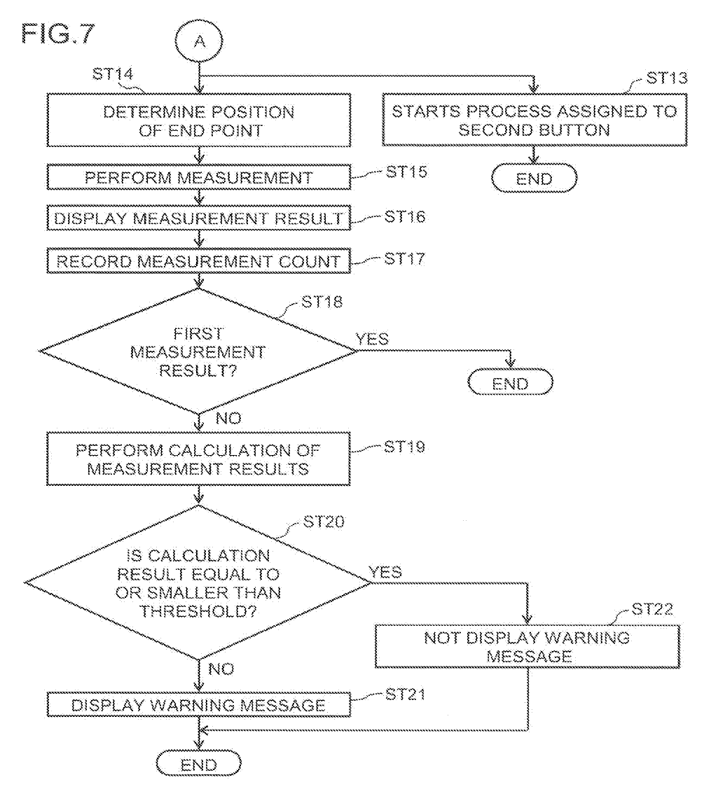

Next, with reference to FIG. 6 and FIG. 7, a description will be given of the process from when the user measures a measurement object until the user is notified of warning that the results contain an abnormal value. FIGS. 6 and 7 are flowcharts illustrating user's operation to measure a measurement object according to the embodiment.

As a prerequisite for the user to start the measurement process, an ultrasound image is generated based on the internal information of the subject acquired by the ultrasound image diagnosis apparatus 1 and displayed on the display 35. The measurement process is performed by using the ultrasound image displayed on the display 35.

First, the user selects a measurement item, thereby the measurement process is started (ST1). While a measurement item is not selected (NO in ST1), the ultrasound image diagnosis apparatus 1 is in standby.

As to the measurement item, for example, as illustrated in FIG. 3, the user selects a desired one of measurement items displayed on the screen 362 of the input circuit 36 and touches the button (YES in ST1).

The control circuit 39 receives a signal from the button of the measurement item touched by the user, and firstly starts the setting of the start point as a measurement start position by the measurement function (ST2). At this time, the control circuit 39 displays calipers suitable for the selected measurement item on the display 35. The user moves the calipers to a desired position as the start point on the screen by using the input circuit 36.

The start point is set at the position where the calipers are arranged as the user presses the first button (ST3). Accordingly, the first button is not pressed while the position of the start point is not determined, (NO in ST3), and the control circuit 39 moves the calipers in response to the user's operation.

The control circuit 39 detects that the user has pressed the first button (YES in ST3), and sets the start point at the position where the calipers are arranged by the user. Thereby, the setting of the start point is completed (ST4). Then, the setting of the end point is started (ST5).

In the same manner as the setting of the start point, the control circuit 39 displays calipers that indicate the end point on the display 35 for setting the end point. The user moves the calipers to a desired position as the end point on the screen by using the input circuit 36.

The end point is also set at the position where the calipers are arranged as the user presses the first button (ST6). Accordingly, the first button is not pressed while the position of the end point is not determined, (NO in ST6), and the control circuit 39 moves the calipers in response to the user's operation.

The control circuit 39 detects that the user has pressed the first button (YES in ST6), and sets the end point at the position where the calipers are arranged by the user. Thereby, the setting of the end point is completed (ST7). When the end point is set, the measurement is started between the start point and the end point (ST8).

The control circuit 39 measures the distance between the start point and the end point by the measurement function, and displays the measurement result on the display 35 (ST9). The control circuit 39 also stores the measurement result in the memory circuit 38. Each time the measurement process is completed, for example, the control circuit 39 increments the value indicating the measurement count by one to record the number of times the measurement process is performed (ST10).

The control circuit 39 checks whether the measurement process has been performed a predetermined number of times set in advance with respect to the measurement item selected (ST11). Having determined that the measurement process has not been performed for the predetermined number of times with respect to the measurement item (NO in ST11), the process returns to step ST2, and the setting of the start point is started to perform the measurement process.

Note that the description is given assuming that the measurement process is performed continuously for the number of times set in advance with respect to each measurement item; however, the measurement processes need not necessarily be performed continuously. The measurement processes is only required to be performed a predetermined number of times in the end.

Besides, the measurement process has been described as being performed with reference to the start point and the end point set by the user using the “first button”. That is, the user sets these positions by pressing the “first button” having the function of setting the start point and the end point. Therefore, when both the start point and the end point are set by using the first button, it can be said that the measurement process is performed after the start point and the end point are each set to a position intended by the user. Therefore, measurement results are hardly likely to contain an abnormal value, and warning is not displayed for the user.

On the other hand, as described above, the position of the end point may be set when, for example, the user presses a button for performing another process than the measurement process without pressing the first button. In this case, it is not obvious whether the position of the end point is set according to the user's clear intention. In other words, the end point may be set to a position not intended by the user. In such a case, it is uncertain whether the measurement process is to be performed accurately, and measurement results may contain an abnormal value.

Incidentally, for example, when the user successively presses buttons of different measurement items and the state transitions before the position of the start point as well as the position of the end point is set, the measurement process is not to be performed.

Accordingly, when the user does not press the first button to set the end point (NO in ST6), the control circuit 39 checks whether the user has pressed the second button (ST12). As described above, the “second button” is a button other than the first button and is a button that the user presses while setting the end point.

When the user has not pressed the second button (NO in ST12), it means that neither the first button nor the second button has been pressed. The process returns to step ST6, and the control circuit 39 checks whether the user has pressed the first button again.

On the other hand, when the user has pressed the second button (YES in ST12), the control circuit 39 starts a process assigned to the second button pressed by the user in response to a signal from the second button (ST13 in FIG. 7). Since another process is started from this point, the measurement process with respect to the measurement item selected, which has been explained so far, ends here.

Meanwhile, when the second button is pressed, the control circuit 39 determines the position of the end point at the position where the calipers are located on the ultrasound image at the time when the second button is pressed (ST14). With this, the start point and the end point have been set, and therefore the control circuit 39 starts measurement between the start point and the end point by the measurement function (ST15).

The control circuit 39 displays a measurement result on the display 35 (ST16). In addition, as the measurement process has been completed, for example, the control circuit 39 increments the measurement count by one to record the number of times the measurement process is performed (ST17). The measurement result is stored in the memory circuit 38.

The control circuit 39 checks whether the measurement result obtained is the first measurement result for the measurement item selected (ST18). Since the end point has been set in response to the pressing of a button other than the first button, the control circuit 39 notifies the user of a warning when the measurement result contains an abnormal value. For that purpose, a calculation process needs to be performed using measurement results of the measurement process. It is checked whether the measurement result is the first one because if there is only one measurement result obtained, the calculation process by the measurement result calculation function cannot be performed.

When at least two measurement results have been obtained, the user can be warned that, for example, one of the measurement results is inappropriate even if it cannot be specified which one indicates an abnormal value.

When the measurement result is the first measurement result for the measurement item selected (YES in ST18), since the process assigned to the second button is already being performed, the measurement process with respect to the measurement item ends here.

Having determined that the measurement result is not the first measurement result for the measurement item selected (NO in ST18), the control circuit 39 performs the calculation of measurement results by the measurement result calculation function (ST19). In the calculation process, as described above, the control circuit 39 may obtain the average of the measurement results and/or the variance thereof according to any one of the two calculation result acquisition methods exemplified above.

The control circuit 39 compares the calculation result of the calculation process with a threshold value by the determination function (ST20). When the calculation result is larger than the threshold value (NO in ST20), for example, a warning message is displayed on the display 35 (ST21). On the other hand, when the calculation result is equal to or smaller than the threshold value (YES in ST20), the warning display is not provided (ST22).

As described above, when the position of the end point is set to a position not intended by the user, and an inappropriate measurement result is obtained, the user can be notified of a warning as to the measurement result.

Specifically, calculation is performed using a plurality of measurement results, and the result thereof is indicated so that the user can be notified of the possibility that the measurement results contain an abnormal value when there is a large variation in the measurement results. It is also possible to indicate which measurement result varies from other measurement results. In this manner, by indicating the variation of measurement results, i.e., indicating that the measurement results contain an abnormal value, the user can be notified of this and make it useful for future determination.

While certain embodiments have been described, these embodiments have been presented by way of example only, and are not intended to limit the scope of the inventions. Indeed, the novel embodiments described herein may be embodied in a variety of other forms; further, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasound image diagnosis apparatus, comprising:
a display configured to display an ultrasound image generated in response to a reflected signal of ultrasound waves transmitted toward a target site to be diagnosed from an ultrasound probe;
an input circuit including a first button that is used to set a start point as a measurement start position and an end point as a measurement end position upon measuring a size of a measurement object on the ultrasound image displayed on the display; and
processing circuitry configured to cause the display to display the ultrasound image, and determine whether the end point is set with the first button or with a second button other than the first button.

2. The ultrasound image diagnosis apparatus of claim 1, wherein the processing circuitry is further configured to:
measure the size of the measurement object indicated by the start point and the end point to obtain a measurement result; and
perform calculation using the measurement result when determining that the end point is set with the second button.

3. The ultrasound image diagnosis apparatus of claim 2, wherein the processing circuitry is further configured to cause the display to display a warning when a result of the calculation exceeds a threshold set in advance.

4. The ultrasound image diagnosis apparatus of claim 2, wherein the processing circuitry is further configured to perform the calculation when a plurality of measurement results have been obtained.

5. The ultrasound image diagnosis apparatus of claim 3, wherein the processing circuitry is further configured to perform the calculation when a plurality of measurement results have been obtained.

6. A medical image processing apparatus, comprising:
a display configured to display a medical image generated based on information on a target site of a subject;
an input circuit including a first button that is used to set a start point as a measurement start position and an end point as a measurement end position upon measuring a size of a measurement object on the ultrasound image displayed on the display; and
processing circuitry configured to:
cause the display to display the medical image;
determine whether the end point is set with the first button or with a second button other than the first button;
measure the size of the measurement object indicated by the start point and the end point to obtain a measurement result;
perform calculation using the measurement result when determining that the end point is set with the second button; and
cause the display to display a warning when a result of the calculation exceeds a threshold set in advance.

7. A computer program product comprising a non-transitory computer-usable medium having computer-readable program codes that, when executed, cause a computer to:
display an ultrasound image generated in response to a reflected signal of ultrasound waves transmitted toward a target site to be diagnosed from an ultrasound probe;
set a start point as a measurement start position and an end point as a measurement end position with a first button on an input circuit upon measuring a size of a measurement object on the ultrasound image displayed on the display
measure the size of the measurement object indicated by the start point and the end point to obtain a measurement result;
determine whether the end point is set with the first button or with a second button other than the first button;
perform calculation using the measurement result when determining that the end point is set with the second button; and
cause the display to display a warning when a result of the calculation exceeds a threshold set in advance.
